(19) United States
(12) Patent Application Publication
(10) Pub. No.: US 2005/0215430 A1
(43) Pub. Date: Sep. 29, 2005

(54) Photocatalyst material

(75) Inventor: Seiichiro Tamai, Osaka (JP)

Correspondence Address: GREENBLUM & BERNSTEIN, P.L.C., 1950 ROLAND CLARKE PLACE, RESTON, VA, 20191, US

(73) Assignee: Matsushita Electric Industrial Co., Ltd., Osaka (JP)

(21) Appl. No.: 11/054,525

(22) Filed: Feb. 10, 2005

(30) Foreign Application Priority Data
Mar. 24, 2004 (JP) 2004-087509

(51) Int. Cl. C07C 1/00

(52) U.S. Cl. 502/350

(57) ABSTRACT

An object of the present invention is to provide a photocatalyst material which fully causes a photocatalyst effect, the material comprising: a phosphorescent layer 110 which is formed above a base substance 100, and has a light-emitting wavelength in the wavelength band of 400 to 700 nm; a translucent layer 120 which is formed above the phosphorescent layer 110 and made of photonic crystal that (i) effectively collects light entering the phosphorescent layer 110, and (iii) effectively collects light entering from the phosphorescent layer 110 into a photocatalyst layer 130; and the photocatalyst layer 130 which is formed above the translucent layer 120, and causes the photocatalyst function using light having the wavelength band of 400 to 700 nm.

### BACKGROUND OF THE INVENTION

(1) Field of the Invention

The present invention relates to a photocatalyst material, in particular, to a photocatalyst material which can fully cause the photocatalyst effect in applied products.

(2) Description of the Related Art

In recent years, antifouling, deodorizing and antibiotic functions of photocatalyst such as titanium oxide have been paid attention to. The above mentioned functions of the photocatalyst are caused by electrons and holes generated by light entering the photocatalyst. The electrons and holes react with oxygen and water on the photocatalyst surface so as to generate active oxygen and OH radical. In other words, the active oxygen and OH radical generated on the photocatalyst surface decompose organic matter in the air.

As a technique which utilizes the above mentioned photocatalyst, for example, there is “illuminating apparatus” (refer to Japanese Laid-Open Patent application No. H11-339521). This illuminating apparatus has the photocatalyst functions. In the above mentioned illuminating apparatus, the cover of the fluorescent light unit (bulb or tube) has the photocatalyst functions, thus antifouling, deodorizing and antibiotic functions are added. More specifically, (i) coating liquid obtained by mixing photocatalyst powder and organic silicide is applied to the cover of the fluorescent light unit, and (ii) a photocatalyst film is formed above the cover of the fluorescent light unit by baking, so as to add the functions of antifouling and the like to the illuminating apparatus.

In the case where the photocatalyst is used for antifouling, deodorizing and antibiotic purposes, the decomposition subject of the photocatalyst includes organic matter which are difficult to be decomposed due to their long molecular chains, such as cigarette tar. In order to decompose the above mentioned organic matter, a lot of active oxygen and OH radicals need to be generated. Thus, it is desirable that the photocatalyst is exposed to light for a long time.

However, in the case where the photocatalyst is used for the illuminating apparatus and the like, the photocatalyst is exposed to light only when the illuminating apparatus is lighted. Thereby, a lot of active oxygen and OH radicals cannot be generated. And, it takes a long time to decompose the organic matter with long molecular chains. Thus, there is a problem that the photocatalyst effect such as antifouling, deodorizing and antibiotic functions cannot be fully caused, by applying the photocatalyst material which only has the photocatalyst functions to the illuminating apparatus.

### SUMMARY OF THE INVENTION

An object of the present invention is to provide a photocatalyst material which can fully cause a photocatalyst effect in the applied products.

In order to achieve the above mentioned object, a photocatalyst material according to the present invention comprises: a phosphorescent layer made of a phosphorescent material; a photocatalyst layer made of a photocatalyst, having a photocatalyst function which is caused by light emitted from the phosphorescent layer; and a translucent layer made of a photonic crystal that effectively collects at least one of light entering the phosphorescent layer and light emitted from the phosphorescent layer into the photocatalyst layer. Here, the photonic crystal may have a flat or concave dispersion surface. Also, the translucent layer may be formed above the phosphorescent layer, and the photocatalyst layer may be formed above the translucent layer. In addition, the phosphorescent layer may be formed above the translucent layer, and the photocatalyst layer is formed above the phosphorescent layer.

Thus, light entering the photocatalyst material is stored in the phosphorescent layer. Even when light does not enter the photocatalyst material, the stored light enters the photocatalyst layer from the phosphorescent layer and, the photocatalyst function can be caused. Thereby, it is possible to realize the photocatalyst material which can fully cause a photocatalyst effect in the applied products. Also, the photonic crystal of the translucent layer has a function such as a lens which effectively collects and takes in light without reflecting the light on the surface (absorption and intake efficiency is improved by more than fifty percent). The light entering the phosphorescent layer and the light emitted from the phosphorescent layer into the photocatalyst layer can be effectively collected. And, light entering the photocatalyst material from outside can effectively enter the phosphorescent layer. In addition, the light emitted from the phosphorescent layer can effectively enter the photocatalyst layer. As a result, the photocatalyst material having a high light usage efficiency can be realized.

Here, the phosphorescent layer may emit light having the wavelength band of 400 to 700 nm, and the photocatalyst layer may have the photocatalyst function which is caused by the light having the wavelength band of 400 to 700 nm.

Thus, the photocatalyst effect can be also caused using illuminating light, and it is possible to realize the photocatalyst material that can be applied to various kinds of products.

Also, the translucent layer may be made of a plurality of photonic crystals which effectively collect light of different wavelength bands. And, the translucent layer may be made of a plurality of photonic crystals which are laminated and effectively collect light of different wavelength bands. The translucent layer may be made of the plurality of photonic crystals which are two-dimensionally arranged and effectively collect light of different wavelength bands. And, the translucent layer may be made of (i) a photonic crystal which effectively collects blue light, (ii) a photonic crystal which effectively collects green light, and (iii) a photonic crystal which effectively collects red light.

Thus, it is not necessary to manufacture a photonic crystal which effectively collect the lights of a wide wavelength band. And, the photonic crystal can be easily manufactured. Thereby, it is possible to realize a photocatalyst material that can be easily manufactured.

Also, the present invention can be applied to an illuminating apparatus comprising (i) a fluorescent light unit on whose surface the photocatalyst material is formed or (ii) a reflector on whose surface the photocatalyst material is formed.

Thus, due to the fluorescent light unit of the illuminating apparatus, active oxygen and OH radical are generated on the photocatalyst surface. And, the active oxygen and OH radical decompose toxic organic matter in the air. Therefore, it is possible to realize an illuminating apparatus which has antifouling, deodorizing and antibiotic functions after turning off the light. Also, a lot of active oxygen and OH radicals can be generated. Thus, it is possible to realize an illuminating apparatus which can decompose the organic matter that is difficult to be decomposed due to its long molecular chain.

As evident from the above mentioned explanation, according to the photocatalyst material of the present invention, it is possible to realize the photocatalyst material which can fully cause the photocatalyst effect in the applied products. Also, it is possible to realize the photocatalyst material which has the high light usage efficiency. In addition, it is possible to realize the photocatalyst material which can be applied to various kinds of products. Moreover, it is possible to realize the photocatalyst material which can be easily manufactured.

Furthermore, according to the present invention, it is possible to realize the illuminating apparatus which has the antifouling, deodorizing and antibiotic functions. Also, it is possible to realize the illuminating apparatus which can cause the photocatalyst effect after the switch-off. And, it is possible to realize the illuminating apparatus which can decompose the organic matter that is difficult to be decomposed due to its long molecular chain.

Consequently, according to the present invention, it is possible to provide the photocatalyst material which can fully cause the photocatalyst effect in the applied products. And, the practical value is extremely high.

### FURTHER INFORMATION ABOUT TECHNICAL BACKGROUND TO THIS APPLICATION

The disclosure of Japanese Patent Application No. 2004-087509 filed on Mar. 24, 2004 including specification, drawings and claims is incorporated herein by reference in its entirety.

### BRIEF DESCRIPTION OF THE DRAWINGS

These and other objects, advantages and features of the invention will become apparent from the following description thereof taken in conjunction with the accompanying drawings that illustrate a specific embodiment of the invention. In the Drawings:

FIG. 1 is a section view showing a structure of a photocatalyst material according to the first embodiment of the present invention;

FIG. 2 is a section view showing an example structure of a translucent layer;

FIGS. 3A, 3B, 3C and 3D are section views for explaining a method for manufacturing a photocatalyst material according to the first embodiment;

FIG. 4 is a section view showing an illuminating apparatus according to the second embodiment; and

FIG. 5 is a section view showing a variation of an illuminating apparatus according to the second embodiment.

### DESCRIPTION OF THE PREFERRED EMBODIMENT(S)

(First Embodiment)

The photocatalyst material according to the first embodiment of the present invention will be explained referring to the drawings.

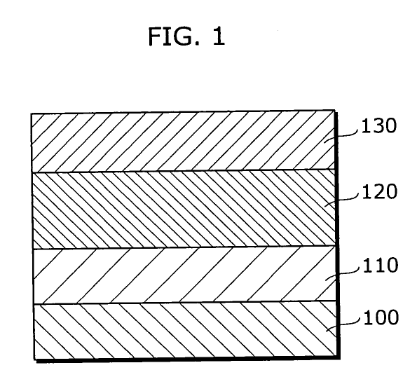

FIG. 1 is a section view showing the photocatalyst material according to the present embodiment.

An object of the photocatalyst material according to the present embodiment is to fully cause the photocatalyst effect in the applied products. The above mentioned photocatalyst material comprises: a phosphorescent layer 110 which is formed above a base substance 100 and emits light having the wavelength band of 400 to 700 nm, in particular, in the wavelength band of 400 to 500 nm; a translucent layer 120 which is (i) formed above the phosphorescent layer 110, and (ii) made of a photonic crystal that effectively collects the light entering from the phosphorescent layer 110 into a photocatalyst layer 130, having the flat dispersion surface; and the photocatalyst layer 130 which (i) is formed above the translucent layer 120, and (ii) causes the photocatalyst functions using the light having the wavelength band of 400 to 700 nm, in particular, in the wavelength band of 400 to 500 nm. The photonic crystal forming the translucent layer 120 may have a concave dispersion surface.

The phosphorescent layer 110 is formed by adhesively bonding a powdery phosphorescent material to the base substance 100 using a binder. Here, for example, a phosphorescent material made of strontium and aluminum oxide compound to which rare earth elements such as europium and dysprosium are doped is used. As the binder, an inorganic resin and an organic resin are used. Here, it is desirable that the binder is transparent so that the light emitted from the phosphorescent material can reach the photocatalyst layer 130.

The photocatalyst layer 130 is formed by adhesively bonding a powdery photocatalyst to the base substance 100 using a binder. Here, as the photocatalyst, for example, a powdery rutile type or anatase type titanium dioxide which (i) impurities are added to or (ii) metal is supported for, so as to cause the photocatalyst functions using visible light is used. Also, as the binder, an inorganic resin and an organic resin are used. Here, it is desirable to use the binder which is difficult to be decomposed by the photocatalysis, such as cement, concrete, gypsum, silicate compound, silica, silicide, silicone resin and fluorocarbon resin. Here, in order to expose the photocatalyst surface so that active oxygen and OH radical generated on the photocatalyst surface can decompose the decomposition subjects in the air, it is desirable that the photocatalyst layer 130 has a plurality of pores. Also, it is desirable that the binder is transparent so that the light which is not absorbed in the photocatalyst among the lights entering the photocatalyst layer 130 can reach the phosphorescent layer 110. Here, the photocatalyst layer 130 may be formed by a physical film forming method such as sputtering or vapor deposition method.

FIG. 2 is a section view showing a structure example of the translucent layer 120.

The translucent layer 120 includes: the first region 200 made of the first refractive index material; and the second region 210 which is a plurality of cylinders that are (i) periodically arranged three-dimensionally in the translucent layer 120, and (ii) made of the second refractive index material that has a different refractive index from the first refractive index material. Here, for example, as the first refractive index material, SiO₂ is used, and as the second refractive index material, Si₃N₄ is used.

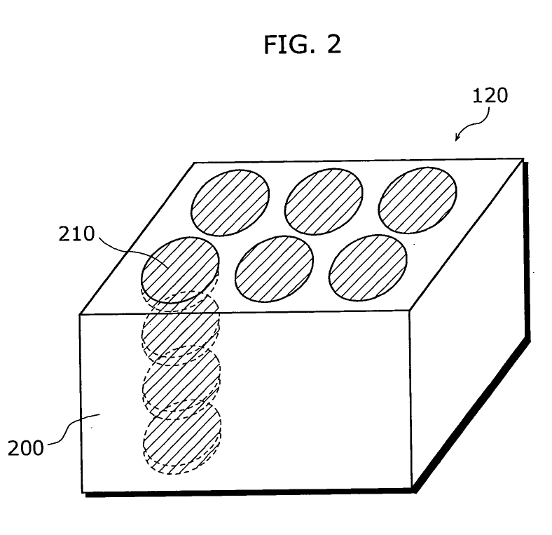

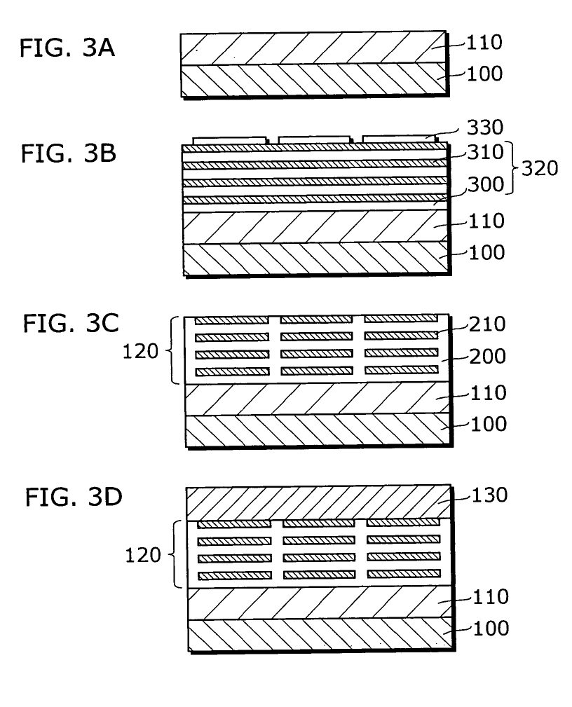

Next, a method for manufacturing a photocatalyst material having the above mentioned structure will be explained using the section views as shown in FIGS. 3A, 3B, 3C and 3D. The same elements in FIG. 1 and FIG. 2 are assigned with the same codes, and the explanation will be omitted here.

First, as shown in FIG. 3A, (i) a phosphorescent material is dispersed to inorganic or organic liquid including a binder, and coating liquid is made, and (ii) after this coating liquid is applied to the surface of the base substance 100, it is dried so as to form the phosphorescent layer 110 on the base substance 100.

Next, as shown in FIG. 3B, after a layer 320 including a layer 300 made of the first refractive index material and a layer 310 made of the second refractive index material is formed a plurality of times on the phosphorescent layer 110, a plurality of cylinder resists 330 are formed above the exposed layer 320.

Next, as shown in FIG. 3C, after the plurality of layers 320 are etched using the resist 330, the first refractive index material is deposited. Thus, the translucent layer 120 is formed.

Next, as shown in FIG. 3D, (i) a photocatalyst is dispersed to inorganic or organic liquid including a binder, and coating liquid is made, and (ii) after this coating liquid is applied to the translucent layer 120, it is dried so as to form a photocatalyst layer 130 above the translucent layer 120. Here, it is desirable to adjust the amount of binder and photocatalyst in the liquid and the drying condition after application so that pores can be formed in the photocatalyst layer 130.

As described above, the photocatalyst material according to the first embodiment comprises: the photocatalyst layer 130 and the phosphorescent layer 110. The light entering the photocatalyst material is stored in the phosphorescent material, thereby the photocatalyst functions can be caused even when the light does not enter the photocatalyst material. Thus, the photocatalyst material according to the present embodiment can fully cause the photocatalyst effect in the applied products.

In addition, the photocatalyst material according to the present embodiment comprises a translucent layer 120 made of photonic crystal that effectively collects the light entering from the phosphorescent layer 110 into the photocatalyst layer 130. The photonic crystal of the translucent layer functions as a lens which effectively collects and takes in the light without reflection on the surface (absorption and intake efficiency is improved by more than fifty percent). The light entering the phosphorescent layer and the light emitted from the phosphorescent layer into the photocatalyst layer can be effectively collected. The light entering the photocatalyst material from outside can effectively enter the phosphorescent layer. Also, the light emitted from the phosphorescent layer can effectively enter the photocatalyst layer. Thus, the photocatalyst material according to the present embodiment has a high light usage efficiency. In such case as described above, the light-condensing rate improves more than fifty percent, compared to the case where the photonic crystal is not used, for example, approximately by one hundred and fifty percent.

Moreover, according to the photocatalyst material of the present embodiment, the photocatalyst layer 130 causes the photocatalyst functions using the light whose wavelength band is from 400 nm to 700 nm. The photocatalyst effect can be caused even with illuminating light. Thus, the photocatalyst material according to the present embodiment can be applied to various products.

In the photocatalyst material according to the present embodiment, (i) the phosphorescent layer 110 is formed above the base substance 100, (ii) the translucent layer 120 is formed above the phosphorescent layer 110, and (iii) the photocatalyst layer 130 is formed above the translucent layer 120. However, in the case where light is emitted from the base substance, (i) the translucent layer may be formed above the base substance, (ii) the phosphorescent layer may be formed above the translucent layer, and (iii) the photocatalyst layer may be formed above the phosphorescent layer. Here, the photonic crystal which effectively collects only the lights entering the phosphorescent layer is used.

Furthermore, according to the photocatalyst material of the present embodiment, the photonic crystal forming the translucent layer 120 effectively collects (i) the light entering the phosphorescent layer 110 and (ii) the light entering from the phosphorescent layer 110 into the photocatalyst layer 130. In other words, one photonic crystal effectively collects all of the lights having the wavelength bands from 400 nm to 700 nm. However, the translucent layer may include a plurality of photonic crystals which effectively collect the lights of different wavelength bands. And, each photonic crystal may effectively collect respective light of different wavelength so that all of the lights having the wavelength bands from 400 nm to 700 nm can be effectively collected. Here, the translucent layer is formed by (i) two-dimensionally arranging or (ii) laminating a plurality of photonic crystals that effectively collect the lights of different wavelength bands. For example, the translucent layer is formed by two-dimensionally arranging or laminating (i) a photonic crystal which effectively collects red light, (ii) a photonic crystal which effectively collects green light and (iii) a photonic crystal which effectively collects blue light. Thereby, it is not necessary to manufacture a photonic crystal which effectively collects all the lights of the wide wavelength band. And, it becomes easy to manufacture a photonic crystal. Thus, the photocatalyst material which is easily manufactured can be realized.

(Second Embodiment)

An illuminating apparatus according to the second embodiment of the present invention will be explained referring to the drawings.

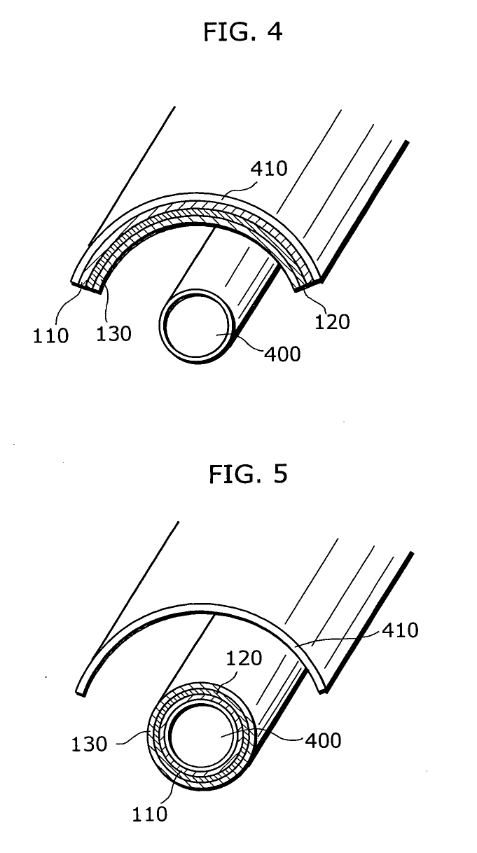

FIG. 4 is a section view showing the illuminating apparatus according to the present embodiment. Here, the same elements as FIG. 1 are assigned with the same codes, and the specific explanation for these will be omitted.

An object of the present embodiment is to realize an illuminating apparatus which has antifouling, deodorizing and antibiotic effects. The illuminating apparatus according to the present embodiment uses the photocatalyst material of the first embodiment.

The illuminating apparatus includes: a fluorescent light unit 400 which emits light; and a reflector 410 which reflects the light of the fluorescent light unit 400. Here, above the reflector 410, a phosphorescent layer 110, a translucent layer 120 and a photocatalyst layer 130 are formed in such order.

As described above, the illuminating apparatus according to the present embodiment includes the photocatalyst layer 130 above the reflector 410. Due to the light entering from the fluorescent light unit into the photocatalyst layer, active oxygen and OH radical are generated on the photocatalyst surface. And, the active oxygen and OH radical decompose hazardous organic matter in the air. Thus, the photocatalyst material according to the present embodiment can realize the illuminating apparatus which has antifouling, deodorizing and antibiotic effects.

Moreover, the illuminating apparatus according to the present embodiment includes: the photocatalyst layer 130 and the phosphorescent layer 110 above the reflector 410. Thereby, light can be provided to the photocatalyst layer after the fluorescent light unit is switched off. Thus, the photocatalyst material according to the present embodiment can realize the illuminating apparatus that causes the photocatalyst effects after the light is switched off. Furthermore, a lot of active oxygen and OH radicals can be generated. Therefore, an illuminating apparatus capable of decomposing organic matter that is difficult to decompose due to its long molecular chain can be realized.

In the illuminating apparatus according to the present embodiment, the phosphorescent layer 110, the translucent layer 120 and the photocatalyst layer 130 are formed in such order above the reflector 410. However, as shown in FIG. 5, the phosphorescent layer 110, the translucent layer 120 and the photocatalyst layer 130 may be formed in such order above the fluorescent light unit 400. Also, the translucent layer, the phosphorescent layer and the photocatalyst layer may be formed in such order above the fluorescent light unit.

Although only some exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention.

### INDUSTRIAL APPLICABILITY

The present invention can be utilized as a photocatalyst material, in particular, as a photocatalyst material used for illuminating apparatuses and exterior walls.

1. A photocatalyst material comprising:
a phosphorescent layer made of a phosphorescent material;
a photocatalyst layer made of a photocatalyst, having a photocatalyst function which is caused by light emitted from the phosphorescent layer; and
a translucent layer made of a photonic crystal that effectively collects at least one of light entering the phosphorescent layer and light emitted from the phosphorescent layer into the photocatalyst layer.

2. The photocatalyst material according to claim 1,
wherein the phosphorescent layer emits light having a wavelength band of 400 to 700 nm, and
the photocatalyst layer has the photocatalyst function which is caused by the light having the wavelength band of 400 to 700 nm.

3. The photocatalyst material according to claim 2,
wherein the translucent layer is made of a plurality of photonic crystals which effectively collect light of different wavelength bands.

4. The photocatalyst material according to claim 3,
wherein the translucent layer includes a plurality of photonic crystals which are laminated and effectively collect light of different wavelength bands.

5. The photocatalyst material according to claim 4,
wherein the translucent layer is made of (i) a photonic crystal which effectively collects blue light, (ii) a photonic crystal which effectively collects green light, and (iii) a photonic crystal which effectively collects red light.

6. The photocatalyst material according to claim 5,
wherein each of the photonic crystal has a flat dispersion surface.

7. The photocatalyst material according to claim 5,
wherein each of the photonic crystal has a concave dispersion surface.

8. The photocatalyst material according to claim 3,
wherein the translucent layer includes a plurality of photonic crystals which are two-dimensionally arranged and effectively collect light of different wavelength bands.

9. The photocatalyst material according to claim 8,
wherein the translucent layer is made of (i) a photonic crystal which effectively collects blue light, (ii) a photonic crystal which effectively collects green light, and (iii) a photonic crystal which effectively collects red light.

10. The photocatalyst material according to claim 9,
wherein each of the photonic crystal has a flat dispersion surface.

11. The photocatalyst material according to claim 9,
wherein each of the photonic crystal has a concave dispersion surface.

12. The photocatalyst material according to claim 1,
wherein the translucent layer is made of a plurality of photonic crystals which effectively collect light of different wavelength bands.

13. The photocatalyst material according to claim 1,
wherein the translucent layer includes a plurality of photonic crystals which are laminated and effectively collect light of different wavelength bands.

14. The photocatalyst material according to claim 1,
wherein the translucent layer includes a plurality of photonic crystals which are two-dimensionally arranged and effectively collect light of different wavelength bands.

15. The photocatalyst material according to claim 1,
wherein each of the photonic crystal has a flat dispersion surface.

16. The photocatalyst material according to claim 1,
wherein each of the photonic crystal has a concave dispersion surface.

17. The photocatalyst material according to claim 1,
wherein the translucent layer is formed above the phosphorescent layer, and
the photocatalyst layer is formed above the translucent layer.

18. The photocatalyst material according to claim 1,
wherein the phosphorescent layer is formed above the translucent layer, and
the photocatalyst layer is formed above the phosphorescent layer.

19. An illuminating apparatus comprising
a fluorescent light unit on whose surface a photocatalyst material is formed, the photocatalyst material including:
a phosphorescent layer made of a phosphorescent material;
a photocatalyst layer made of a photocatalyst, having a photocatalyst function which is caused by light emitted from the phosphorescent layer; and
a translucent layer made of a photonic crystal that effectively collects at least one of light entering the phosphorescent layer and light emitted from the phosphorescent layer into the photocatalyst layer.

20. An illuminating apparatus comprising
a reflector on whose surface a photocatalyst material is formed, the photocatalyst material including:
a phosphorescent layer made of a phosphorescent material;
a photocatalyst layer made of a photocatalyst, having a photocatalyst function which is caused by light emitted from the phosphorescent layer; and
a translucent layer made of a photonic crystal that effectively collects at least one of light entering the phosphorescent layer and light emitted from the phosphorescent layer into the photocatalyst layer.
